(19) United States
(12) Patent Application Publication
(10) Pub. No.: US 2011/0250255 A1
(43) Pub. Date: Oct. 13, 2011

(54) DRUG COATED STENT WITH ENDOSOME-DISRUPTING CONJUGATE

(75) Inventors: Ed PARSONAGE, St. Paul, MN (US); John KREMER, Sun Prairie, WI (US)

(73) Assignee: BOSTON SCIENTIFIC SCIMED, INC., Maple Grove, MN (US)

(21) Appl. No.: 13/078,686

(22) Filed: Apr. 1, 2011

Related U.S. Application Data
(63) Continuation of application No. 12/329,803, filed on Dec. 8, 2008, now abandoned.
(60) Provisional application No. 61/012,381, filed on Dec. 7, 2007.

(51) Int. Cl. A61F 2/82; A61K 38/08; A61K 38/10; A61P 23/00; A61P 37/06; A61P 7/02; A61P 35/00; A61P 29/00; A61K 31/436; A61K 38/16

(52) U.S. Cl. 424/423; 514/291; 514/1.1; 514/21.6; 514/21.2; 514/21.3

(57) ABSTRACT

A stent is provided with a drug-eluting layer disposed on at least a portion of its surface, wherein the drug-eluting layer comprises an endosome-disrupting agent and a pharmaceutical agent. In an embodiment, the endosome-disrupting agent, when taken up through endocytosis into living cells, causes lysis of endosomes containing the endosome-disrupting agent. The pharmaceutical agent can accompany the endosome-disrupting agent into the living cells.

### CROSS REFERENCE TO RELATED APPLICATION

The present application claims priority to U.S. provisional application Ser. No. 61/012,381 filed Dec. 7, 2007, the disclosure of which is incorporated herein by reference in its entirety.

### BACKGROUND

The systemic administration of drug agents, such as by transoral or intravenous means, treats the body as a whole even though the disease to be treated may be localized. In such a case, systemic administration may not be desirable because the drug agents may have unwanted effects on parts of the body which are not to be treated, or because treatment of the diseased part of the body requires a high concentration of drug agent that may not be achievable by systemic administration. It is therefore often desirable to administer drug agents at localized sites within the body. Common examples include cases of localized disease (e.g., heart disease) or occluded body lumens.

### DESCRIPTION

For the following defined terms, these definitions shall be applied, unless a different definition is given in the claims or elsewhere in this specification.

As used in this specification and the appended claims, the singular forms “a”, “an”, and “the” include plural referents unless the content clearly dictates otherwise.

As used in this specification and the appended claims, the term “or” is generally employed in its sense including “and/or” unless the content clearly dictates otherwise.

Standard single letter and three letter abbreviations are used herein to refer to naturally-occurring and non-naturally occurring amino acids either individually or connected within a polypeptide chain.

As used herein a “layer” of a given material is a region of that material whose thickness is small compared to both its length and width. As used herein a layer need not be planar, for example, taking on the contours of an underlying substrate. A layer can be discontinuous, providing only partial coverage of the underlying substrate. Terms such as “film,” “layer” and “coating” are used interchangeably herein.

Soluble or solubility refers to the ability of a solid substance when blended with a liquid solvent to lose its crystalline form and become molecularly or ionically dispersed in the solvent. Solids vary from 0 to 100% in their degree of solubility in the solvent. Physiologically soluble refers to solubility where the liquid solvent is a biological fluid, e.g., blood, interstitial fluid, cytoplasm, preferably of a living cell or organism.

Biological conditions refers to the conditions (e.g., aqueous medium, temperature, ionic strength, pH, inside a living organism or inside or surrounding a living cells in vivo. Biological conditions for purposes of the present disclosure are, unless otherwise noted, aqueous solution, 30-40° C., pH 7.0-7.6. Biological conditions of normal human blood 7.35-7.45 are aqueous solution, about 37° C., pH 7.3-7.5.

“Alkyl” refers to a lower alkyl group, a haloalkyl group, an alkenyl group, an alkynyl group, a bridged cycloalkyl group, a cycloalkyl group or a heterocyclic ring, as defined herein.

“Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein.

Exemplary haloalkyl groups include trifluoromethyl, chloromethyl, 2-bromobutyl, 1-bromo-2-chloro-pentyl, and the like.

“Alkenyl” refers to a branched or straight chain C2-C10 hydrocarbon (preferably a C2-C8 hydrocarbon, more preferably a C2-C6 hydrocarbon) which can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Alkynyl” refers to an unsaturated acyclic C₂-C₁₀ hydrocarbon (preferably a C₂-C₈ hydrocarbon, more preferably a C₂-C₆ hydrocarbon) which can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo[3.3.0]octane, 7-oxabycyclo[2.2.1]heptyl, 8-azabicyclo[3,2,1]oct-2-enyl and the like.

“Cycloalkyl” refers to a saturated or unsaturated cyclic hydrocarbon comprising from about 3 to about 8 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo and nitro. Exemplary cycloalkyl groups include cyclopropyl, cyclobutyl, cyclopentyl, cyclohexyl, cyclohexenyl, cycloheptenyl, and the like.

“Heterocyclic ring or group” refers to a saturated, unsaturated, cyclic or aromatic or polycyclic hydrocarbon group having about 3 to about 12 carbon atoms (preferably about 4 to about 6 carbon atoms) where 1 to about 4 carbon atoms are replaced by one or more nitrogen, oxygen and/or sulfur atoms. Sulfur maybe in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylamino, dialkylamino, arylamino, diarylamino, alkylarylamino, hydroxy, oxo, thial, halo, carboxyl, carboxylic ester, alkylcarboxylic acid, alkylcarboxylic ester, aryl, arylcarboxylic acid, arylcarboxylic ester, amidyl, ester, carboxamido, alkylcarboxamido, arylcarboxamido, sulfonic acid, sulfonic ester, sulfonamido and nitro. Exemplary heterocyclic groups include pyrrolyl, 3-pyrrolinyl, 4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrhydrofuranyl, tetrazolyl, 2-pyrrolinyl, 3-pyrrolinyl, pyrrolindinyl, oxazolindinyl 1,3-dioxolanyl, 2,6-dioxabicyclo[3,3,0]octanyl, 2-imidazonlinyl, imidazolindinyl, 2-pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, quinolinyl, and the like.

Various embodiments of the present invention will be described in detail with reference to the drawings, wherein like reference numerals represent like parts throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

All publications and patent applications in this specification are indicative of the level of ordinary skill in the art to which this invention pertains and are incorporated herein by reference in their entireties.

### BRIEF DESCRIPTION OF THE DRAWINGS

FIG. 1 is a flow-chart of the reaction scheme of DCC (Dicyclohexyl Carbodiimide) with the carboxylate group of PG (poly (L-glutamic acid)) and the hydroxyl group of PTx (paclitaxel), carried out in N,N-dimethylformamide (DMF) to form an ester, as described below in an example.

### DETAILED DESCRIPTION OF THE INVENTION

The present disclosure provides an implantable medical device having a drug-eluting coating including an endosome-disrupting agent. Generally, an implantable medical device according to the present disclosure has a coating including an endosome-disrupting agent and a therapeutic agent, disposed on a surface of the implantable device. The endosome-disrupting agent is a compound which has a finite solubility under biologically relevant conditions and when taken up by living cells, causes lysis of endosomes containing the endosome-disrupting agent. Typically, the endosome-disrupting agent accompanies a therapeutic agent into the living cells. In some instances, a stent comprising a coating on a surface thereof, the coating provided by a step of binding endosome-disrupting agents onto at least a portion of a surface of the stent, is provided. The present disclosure also provides a method for forming a layer of endosome-disrupting agent on at least a portion of a substrate surface, wherein the substrate is an implantable medical device.

Endosome-Disrupting Agent

The endosome-disrupting agent of the present disclosure is a non-toxic compound which has finite solubility under biologically relevant conditions and when taken up through endocytosis into living cells, causes lysis of endosomes containing the endosome-disrupting agent, thereby releasing the contents of the endosome into the cytosol. The endosome-disrupting agent is generally soluble at biological conditions of blood, e.g., human artial blood, but is sensitive to change from neutral pH to acidic pH. Typically, an endosome-disrupting agent of the present disclosure undergoes a phase transition as a result of exposure to an acidic environment, such as is generally found within an endosome. Typically, a pH change from about pH 7 to about pH 5, will cause a phase transition of the endosome-disrupting agent. For example a change in pH from pH 7.3-7.5 (blood pH) to a pH less than pH 5.5. In some cases, a pH change from about pH 7.3-7.5 to less than or equal to pH 6.0 is sufficient to cause a phase transition.

A phase transition of the endosome-disrupting agent as used herein is a change from a “native state” of the endosome-disrupting agent at neutral pH to an “active state” at acidic pH. Transition to an “active state” of the endosome-disrupting agent renders the endosome-disrupting agent able to disrupt the endosomal membrane, thereby releasing the endosome contents into the cytosol of the cell. Example means for phase transition by which endosome-disrupting agents may rupture the endosome are: desolvation of the endosome-disrupting agent, loss of charge of the endosome-disrupting agent, and/or intercalation of the endosome-disrupting agent into the endosome membrane. In some instances, suitable endosome-disrupting agents according to the present disclosure may be soluble at about pH 7.4, but have increased hydrophobicity at pH 6.0 or below, such that the endosome-disrupting agent enters and disrupts the endosome membrane. In other cases, suitable endosome-disrupting agents, under acidic conditions, coordinate into endosome membranes, thereby forming higher order structures (e.g., secondary, tertiary, quaternary), such as pores or channels. The higher order structures in the endosome membrane may allow egress of therapeutic agents from the endosome, or entry of cytosol components (e.g., ions and fluid) leading to rupture of the endosome, or both.

A phase transition has occurred when at least a portion of the endosome-disrupting agent present in an endosome exhibits a change in state. A phase transition of at least a portion of an endosome-disrupting agent is considered to have occurred when an endosome ruptures after an endosome-disrupting agent has been taken-up by the cell containing the endosome.

Suitability for an endosome-disrupting agent may be determined by incubating living cells with an endosome-disrupting agent accompanied by a therapeutic agent or non-therapeutic marker molecule in a medium of neutral pH. After a suitable incubation time, the cells are removed from the medium, washed, and analyzed by known methods to detect the presence within the cytosol and/or endosomes for the endosome-disrupting agent or therapeutic agent or non-therapeutic molecule. An endosome-disrupting agent is suitable if the detected molecule, i.e. endosome-disrupting agent, therapeutic agent or non-therapeutic agent marker molecule, is found in the cytosol. Non-therapeutic marker molecules include any molecule that may be readily detected. Suitable marker molecules include fluorescent molecules, radioactive molecules, etc. . . . . The uptake and location of the endosome-disrupting agent, and therapeutic agent or non-therapeutic marker molecules in the cell are time dependent and may vary by cell type. Incubation time should be at least 30 minutes, typically 1 hour or more. Incubation may be as long as 24 hours, 36 hours, or 48 hours.

Suitable endosome-disrupting agents generally include physiologically soluble polymers containing ionizable groups which are sensitive to pH changes occurring under biological conditions. Typically, the physiologically soluble polymers do not show membrane disruptive effects in solution having pH greater than 7.0, and do show pH dependent membrane disruptive effects in solution having pH less than 6.0.

In some instances, the physiologically soluble polymer of the endosome-disrupting agent contains or is derivatized with carboxylic acid functional groups. The amount of carboxylic acid functional groups on the endosome-disrupting agent should be sufficient to produce the desired pH dependent behavior, i.e. phase transition. The amount of carboxylic acid functional groups may range from 1-100%. The physiologically soluble polymer may be a homopolymer or co-polymer including one or more, same or different, monomer units of formula I:

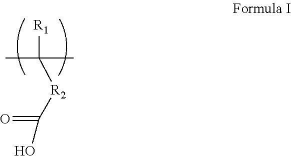

wherein R1 is H or C₁ to C₁₀ alkyl, optionally substituted with O, S, N, or one or more halogens, and R2 is C₁ to C₁₀ lower alkyl, optionally substituted with O, S, N, or one or more halogens. Often, Formula I includes monomer units wherein R1 is H or C₁ to C₈ alkyl, optionally substituted with O, S, N, or one or more halogens, and R2 is C₁ to C₈ lower alkyl, optionally substituted with O, S, N, or one or more halogens. Formula I sometimes includes monomer units wherein R1 is H or C₁ to C₆ alkyl, optionally substituted with O, S, N, or one or more halogens, and R2 is C₁ to C₆ lower alkyl, optionally substituted with O, S, N, or one or more halogens.

A monomer unit of Formula I, wherein R1 is H or C₁ to C₁₀ alkyl, includes wherein R1 is H or a C₁ to C₁₀ lower alkyl group, an C₁ to C₁₀ alkynyl group, a C₁ to C₁₀ bridged cycloalkyl group, a C₁ to C₁₀ cycloalkyl group or a C₁ to C₁₀ heterocyclic ring, any of which may be optionally substituted with O, S, N, or one or more halogens; and R2 is C₁ to C₁₀ lower alkyl, optionally substituted with O, S, N, or one or more halogens. Formula I may include monomer units wherein R1 is H or C₁ to C₈ alkyl, optionally substituted with O, S, N, or one or more halogens, and R2 is C₁ to C₈ lower alkyl, optionally substituted with O, S, N, or one or more halogens. Formula I may include wherein R1 is H or C₁ to C₆ alkyl, optionally substituted with O, S, N, or one or more halogens, and R2 is C₁ to C₆ lower alkyl, optionally substituted with O, S, N, or one or more halogens.

In some instances, the physiologically soluble polymer includes monomer units of Formula I, wherein R1 and R2 are individually selected from H or C₁ to C₁₀ lower alkyl. Occasionally, the physiologically soluble polymer includes monomer units of Formula I, wherein R1 and R2 are individually selected from H or C₁ to C₈ lower alkyl. Occasionally, the physiologically soluble polymer includes monomer units of Formula I, wherein R1 and R2 are individually selected from H or C₁ to C₆ lower alkyl.

In any of the above physiologically soluble polymers including monomers of Formula I, R1 and R2 may be optionally substituted with one or more halogens.

In some cases, other monomer units in addition to those of Formula I, may be included into the endosome-disrupting agent to enhance or modify solubility as a function of pH.

Specific examples of physiologically soluble polymers which arecarboxylic acid functionalized polymers suitable for use as endosome-disrupting agents include, but are not limited to, polyacrylic acid, polymethacrylic acid, polyethylacrylic acid, polypropylacrylic acid, and hydrolyzed polystryrene-co-maleic anhydride copolymer, n-isopropyl acrylamide methacrylic acid copolymers, polyglutamic acid and polyaspartic acid, co-polymers and derivatives thereof.

A physiologically soluble polymer of the endosome-disrupting agent may alternatively or additionally contain or be derivatized with amine functional groups. A physiologically soluble polymer containing monomers having at least one amine functional group, for use as an endosome-disrupting agent, should contain a sufficient number of monomers having at least one amine functional group to produce the desired pH dependent behavior, i.e. phase transition. The number of amine monomers of a physiologically soluble polymer may be from 1-100%. The physiologically soluble polymer may be a homopolymer or co-polymer including one or more, same or different, monomer units of the Formula II:

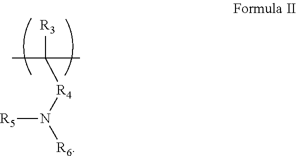

wherein R3 is H or C₁ to C₁₀ alkyl, optionally substituted with O, S, N, or one or more halogens; R4 is absent or C₁ to C₁₀ lower alkyl, optionally substituted with O, S, N, or one or more halogens; R5 and R6 are individually selected from H or C₁ to C₁₀ alkyl, optionally substituted with O, S, N, or one or more halogens. Furthermore, the pendant R5 group may be hydrogen or an organic functionality containing from 1 to 10 carbon atoms. Furthermore, the spacer R6 group may also be an organic functionality having from 0 to 10 carbon atoms. In addition, the amine pendant groups R5 and R6 may also be hydrogen or an organic functionality having from 0 to 10 carbon atoms.

Often, Formula II includes monomer units wherein R3 is H or C₁ to C₈ alkyl, optionally substituted with O, S, N, or one or more halogens, and R4 is C₁ to C₈ lower alkyl, optionally substituted with O, S, N, or one or more halogens. Formula II sometimes includes monomer units wherein R3 is H or C₁ to C₆ alkyl, optionally substituted with O, S, N, or one or more halogens, and R4 is C₁ to C₆ lower alkyl, optionally substituted with O, S, N, or one or more halogens.

A monomer unit of Formula II, wherein R3 is H or C₁ to C₁₀ alkyl, includes wherein R3 is H or a C₁ to C₁₀ lower alkyl group, an C₁ to C₁₀ alkynyl group, a C₁ to C₁₀ bridged cycloalkyl group, a C₁ to C₁₀ cycloalkyl group or a C₁ to C₁₀ heterocyclic ring, any of which may be optionally substituted with O, S, N, or one or more halogens; and R4 is C₁ to C₁₀ lower alkyl, optionally substituted with O, S, N, or one or more halogens. Formula II may include monomer units wherein R3 is H or C₁ to C₈ alkyl, optionally substituted with O, S, N, or one or more halogens, and R4 is C₁ to C₈ lower alkyl, optionally substituted with O, S, N, or one or more halogens. Formula II may include wherein R3 is H or C₁ to C₆ alkyl, optionally substituted with O, S, N, or one or more halogens, and R4 is C₁ to C₆ lower alkyl, optionally substituted with O, S, N, or one or more halogens.

In some instances, the physiologically soluble polymer includes monomer units of Formula II, wherein R3 and R4 are individually selected from H or C₁ to C₁₀ lower alkyl. Occasionally, the physiologically soluble polymer includes monomer units of Formula II, wherein R3 and R4 are individually selected from H or C₁ to C₈ lower alkyl. Occasionally, the physiologically soluble polymer includes monomer units of Formula II, wherein R3 and R4 are individually selected from H or C₁ to C₆ lower alkyl.

In any of the above physiologically soluble polymers including monomers of Formula II, R3 and R4 may be optionally substituted with one or more halogens.

In some cases, other monomer units may be included into the endosome-disrupting agent to enhance solubility as a function of pH.

Specific examples of amine functionalized physiolocially soluble polymers suitable for endosome-disrupting agent include polyethylene imine (PEI), polylysine, poly(amidoamine) dendrimers poly(L-lactide-co-L-lysine), poly(serine ester), poly(4-hydroxy-L-proline ester), poly[alpha.-(4-aminobutyl)-L-glycolic acid]. Poly(4-hydroxy-L-proline ester), poly[.alpha.-(4-aminobutyl)-L-glycolic acid], and poly(beta-amino esters).

Amine containing polymer may also include known endosome disruptive peptides such as GALA, KALA and melittin. In an embodiment of the present disclosure, the endosome-disrupting agent is a peptide which in integrated into the endosomal membrane in acidic environments. In some instances, the peptidyl endosome-disrupting agent adopts a secondary or tertiary protein structure which creates an opening, e.g., a pore, in the endosome membrane.

GALA is a synthetic pore-forming peptide having a repeated peptide motif ‘EXLA’ which exists as a random coil in aqueous solutions above pH 5 and forms an amipathic α-helix in solution at pH 5 and below. Generally, suitable GALA peptides include at least 10 amino acids, typically 20-100 amino acids. One example of a GALA peptide is WEAALAEALAEALAEHLAEALAEALEALAA (SEQ ID NO: 1), also referred to as GALA 30. GALA peptides solvated in aqueous solution at neutral pH, do not form α-helix because of the electrostatic repulsions between the glutamic acid residues. However, at pH 5, the neutralization of the glutamic acid residues promotes the formation of an amphipathic α-helix and GALA binding to lipid bilayers, such as endosome membrane. In the optimal pH range of 5 and below, GALA induces the leakage of the endosome membranes and rapid changeover in membrane structure (flip-flop of phospholipids). GALA peptides of various sizes may be synthesized by known methods, including those of Nicol et al. (1999) Biophys J, 76:2121-2141, incorporated herein by reference.

KALA is another example of a synthetic pore-forming peptide having demonstrated membrane-disrupting properties. KALA is a cationic peptide with a major repeat sequence of ‘KALA.’ KALA exists as a random coil in aqueous solutions above pH 5 and forms an amipathic α-helix in solution at pH 5 and below. One example of a GALA peptide is Trp-Glu-Ala-Lys-Leu-Ala-Lys-Ala-Leu-Ala-Lys-Ala-Leu-Ala-Lys-His-Leu-Ala-Lys-Ala-Leu-Ala-Lys-Ala-Leu-Ala-Lys-Ala-Leu-Lys-Ala-Cys-Glu-Ala (SEQ ID NO:2).

JTS1 is yet another example of a synthetic pore-forming peptide having demonstrated membrane-disrupting properties.

Drug Conjugated to Endosome-Disrupting Agent

Typically, the endosome-disrupting agent is accompanied by one or more therapeutic agents. Often, a endosome-disrupting agent is attached to a therapeutic agent. On occasion, an endosome-disrupting agent is attached to one or more therapeutic agents. Attachment of the endosome-disrupting agent may be directly to a therapeutic agent or indirect, for example via a linker. Alternatively, an endosome-disrupting agent is not fixedly attached, but instead mixed with one or more therapeutic agents for concurrent delivery, for example concurrent diffusion from a stent.

In general, endosome-disrupting agents may be joined to each other and to therapeutic agents by linkers. Linkers may provide flexibility, secondary or higher level structure (e.g., α-helix) and reactive sites for attachment to therapeutic agents. On occasion, linkers are cleavable in vivo.

Example linkers suitable for use as described herein may be selected from any alkane, alkene, or aromatic molecules, any of which may be hetero-substituted with N, S, or O and combinations thereof, which are capable of attachment. Often, linkers are selected from polyethylene glycol, polyethyleneoxide, and polypeptides of naturally-occurring and non-naturally occurring amino acids.

Therapeutic Agents

“Therapeutic agents,” “biologically active agents,” “drugs,” “pharmaceutically active agents,” “pharmaceutically active materials,” and other related terms may be used interchangeably herein. A wide variety of therapeutic agents can be employed in conjunction with the implantable medical devices disclosed herein including those used for the treatment of a wide variety of diseases and conditions (i.e., the reduction or elimination of symptoms associated with a disease or condition, or the substantial or complete elimination of a disease or condition). Numerous therapeutic agents are listed below.

The therapeutic agent may be any medicinal agent which may provide a desired effect. Suitable therapeutic agents include pharmaceuticals, genetic materials, and biological materials. For instance, in some embodiments, the therapeutic agent may include a drug which may be used in the treatment of restenosis. Some suitable therapeutic agents which may be loaded but are not necessarily limited to, antibiotics, antimicrobials, antiproliferatives, antineoplastics, antioxidants, endothelial cell growth factors, thrombin inhibitors, immunosuppressants, anti-platelet aggregation agents, collagen synthesis inhibitors, therapeutic antibodies, nitric oxide donors, antisense oligonucleotides, wound healing agents, therapeutic gene transfer constructs, peptides, proteins, extracellular matrix components, vasodialators, thrombolytics, anti-metabolites, growth factor agonists, antimitotics, steroidal and non-steroidal anti-inflammatory agents, angiotensin converting enzyme (ACE) inhibitors, free radical scavengers, and anticancer chemotherapeutic agents.

In some embodiments, the therapeutic agent is useful for inhibiting cell proliferation, contraction, migration, hyperactivity, or addressing other conditions. The term “therapeutic agent” encompasses pharmaceuticals, genetic materials, and biological materials. Non-limiting examples of suitable therapeutic agents include heparin, heparin derivatives, urokinase, dextrophenylalanine proline arginine chloromethylketone (PPack), enoxaprin, angiopeptin, hirudin, acetylsalicylic acid, tacrolimus, everolimus, rapamycin (sirolimus), amlodipine, doxazosin, glucocorticoids, betamethasone, dexamethasone, prednisolone, corticosterone, budesonide, sulfasalazine, rosiglitazone, mycophenolic acid, mesalamine, paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, lidocaine, bupivacaine, ropivacaine, D-Phe-Pro-Arg chloromethyl ketone, platelet receptor antagonists, anti thrombin antibodies, anti platelet receptor antibodies, aspirin, dipyridamole, protamine, hirudin, prostaglandin inhibitors, platelet inhibitors, trapidil, liprostin, tick antiplatelet peptides, 5-azacytidine, vascular endothelial growth factors, growth factor receptors, transcriptional activators, translational promoters, antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin, cholesterol lowering agents, vasodilating agents, agents which interfere with endogenous vasoactive mechanisms, antioxidants, probucol, antibiotic agents, penicillin, cefoxitin, oxacillin, tobranycin, angiogenic substances, fibroblast growth factors, estrogen, estradiol (E2), estriol (E3), 17-beta estradiol, digoxin, beta blockers, captopril, enalopril, statins, steroids, vitamins, taxol, paclitaxel, 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, 2′-O-ester with N-(dimethylaminoethyl) glutamide hydrochloride salt, nitroglycerin, nitrous oxides, nitric oxides, antibiotics, aspirins, digitalis, estrogen, estradiol and glycosides. In one embodiment, the therapeutic agent is taxol (e.g., Taxol®), or its analogs or derivatives. In another embodiment, the therapeutic agent is paclitaxel. In yet another embodiment, the therapeutic agent is an antibiotic such as erythromycin, amphotericin, rapamycin, adriamycin, etc.

The term “genetic materials” means DNA or RNA, including, without limitation, DNA/RNA encoding of a useful protein stated below, intended to be inserted into a human body including viral vectors and non-viral vectors.

The term “biological materials” include cells, yeasts, bacteria, proteins, peptides, cytokines and hormones. Examples for peptides and proteins include vascular endothelial growth factor (VEGF), transforming growth factor (TGF), fibroblast growth factor (FGF), epidermal growth factor (EGF), cartilage growth factor (CGF), nerve growth factor (NGF), keratinocyte growth factor (KGF), skeletal growth factor (SGF), osteoblast-derived growth factor (BDGF), hepatocyte growth factor (HGF), insulin-like growth factor (IGF), cytokine growth factors (CGF), platelet-derived growth factor (PDGF), hypoxia inducible factor-1 (HIF-1), stem cell derived factor (SDF), stem cell factor (SCF), endothelial cell growth supplement (ECGS), granulocyte macrophage colony stimulating factor (GM-CSF), growth differentiation factor (GDF), integrin modulating factor (IMF), calmodulin (CaM), thymidine kinase (TK), tumor necrosis factor (TNF), growth hormone (GH), bone morphogenic protein (BMP) (e.g., BMP-2, BMP-3, BMP-4, BMP-5, BMP-6 (Vgr-1), BMP-7 (PO-1), BMP-8, BMP-9, BMP-10, BMP-11, BMP-12, BMP-14, BMP-15, BMP-16, etc.), matrix metalloproteinase (MMP), tissue inhibitor of matrix metalloproteinase (TIMP), cytokines, interleukin (e.g., IL-1, IL-2, IL-3, IL-4, IL-5, IL-6, IL-7, IL-8, IL-9, IL-10, IL-11, IL-12, IL-15, etc.), lymphokines, interferon, integrin, collagen (all types), elastin, fibrillins, fibronectin, vitronectin, laminin, glycosaminoglycans, proteoglycans, transferrin, cytotactin, cell binding domains (e.g., RGD), and tenascin. Currently preferred BMP's are BMP-2, BMP-3, BMP-4, BMP-5, BMP-6, BMP-7. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include progenitor cells (e.g., endothelial progenitor cells), stem cells (e.g., mesenchymal, hematopoietic, neuronal), stromal cells, parenchymal cells, undifferentiated cells, fibroblasts, macrophage, and satellite cells.

Other non-genetic therapeutic agents include:
anti-thrombogenic agents such as heparin, heparin derivatives, urokinase, and PPack (dextrophenylalanine proline arginine chloromethylketone);
anti-proliferative agents such as enoxaprin, angiopeptin, or monoclonal antibodies capable of blocking smooth muscle cell proliferation, hirudin, acetylsalicylic acid, tacrolimus, everolimus, amlodipine and doxazosin;
anti-inflammatory agents such as glucocorticoids, betamethasone, dexamethasone, prednisolone, corticosterone, budesonide, estrogen, sulfasalazine, rosiglitazone, mycophenolic acid and mesalamine;
anti-neoplastic/anti-proliferative/anti-miotic agents such as paclitaxel, 5-fluorouracil, cisplatin, vinblastine, vincristine, epothilones, methotrexate, azathioprine, adriamycin, mutamycin, endostatin, angiostatin, thymidine kinase inhibitors, cladribine, taxol and its analogs or derivatives;
anesthetic agents such as lidocaine, bupivacaine, and ropivacaine;
anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors, antiplatelet agents such as trapidil or liprostin and tick antiplatelet peptides;
DNA demethylating drugs such as 5-azacytidine, which is also categorized as a RNA or DNA metabolite that inhibit cell growth and induce apoptosis in certain cancer cells;
vascular cell growth promoters such as growth factors, vascular endothelial growth factors (VEGF, all types including VEGF-2), growth factor receptors, transcriptional activators, and translational promoters;
vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin;
cholesterol-lowering agents; vasodilating agents; and agents which interfere with endogenous vasoactive mechanisms;
anti-oxidants, such as probucol;
antibiotic agents, such as penicillin, cefoxitin, oxacillin, tobranycin, macrolides such as rapamycin (sirolimus) and everolimuns;
angiogenic substances, such as acidic and basic fibroblast growth factors, estrogen including estradiol (E2), estriol (E3) and 17-beta estradiol; and
drugs for heart failure, such as digoxin, beta-blockers, angiotensin-converting enzyme (ACE) inhibitors including captopril and enalopril, statins and related compounds. Preferred biologically active materials include anti-proliferative drugs such as steroids, vitamins, and restenosis-inhibiting agents. Preferred restenosis-inhibiting agents include microtubule stabilizing agents such as Taxol®, paclitaxel (i.e., paclitaxel, paclitaxel analogues, or paclitaxel derivatives, and mixtures thereof). For example, derivatives suitable for use in the present invention include 2′-succinyl-taxol, 2′-succinyl-taxol triethanolamine, 2′-glutaryl-taxol, 2′-glutaryl-taxol triethanolamine salt, 2′-O-ester with N-(dimethylaminoethyl) glutamine, and 2′-O-ester with N-(dimethylaminoethyl) glutamide hydrochloride salt.

Other preferred therapeutic agents include nitroglycerin, nitrous oxides, nitric oxides, antibiotics, aspirins, digitalis, estrogen derivatives such as estradiol and glycosides.

Implantable Medical Devices (Stents) for Delivery of Drug Conjugated to Endosome-Disrupting Agent

Implantable medical devices are provided which include a substrate having a surface, wherein at least a portion of the substrate surface may have endosome-disrupting agent and therapeutica agent disposed thereof. Typically, implantable medical devices are provided which include a substrate having a surface, wherein at least a portion of the substrate surface may have endosome-disrupting agent conjugated to at least one therapeutic agent disposed thereon.

Examples of implantable medical device include, for example, stents (including coronary vascular stents, peripheral vascular stents, cerebral, urethral, ureteral, biliary, tracheal, gastrointestinal and esophageal stents), stent coverings, stent grafts, vascular grafts, abdominal aortic aneurysm (AAA) devices (e.g., AAA stents, AAA grafts), vascular access ports, dialysis ports, catheters (e.g., urological catheters or vascular catheters such as balloon catheters and various central venous catheters), balloons, filters (e.g., vena cava filters and mesh filters for distil protection devices), embolization devices including cerebral aneurysm filler coils, septal defect closure devices, myocardial plugs, patches, pacemakers, lead coatings including pacemaker leads, defibrillation leads and coils, ventricular assist devices including left ventricular assist hears and pumps, total artificial hearts, shunts, valves including heart valves and vascular valves, anastomosis clips and rings, cochlear implants, tissue bulking devices, and tissue engineering scaffolds for cartilage, bone, skin and other in vivo tissue regeneration, sutures, suture anchors, tissue staples and ligating clips at surgical sites, cannulae, metal wire ligatures, urethral slings, hernia “meshes”, artificial ligaments, orthopedic prosthesis such as bone grafts, bone plates, fins and fusion devices, joint prostheses, orthopedic fixation devices such as interference screws in the ankle, knee, and hand areas, tacks for ligament attachment and meniscal repair, dental implants, or other devices that are implanted into the body in contact with endothelium.

Medical devices having a may have endosome-disrupting agent conjugated to at least one therapeutic agent disposed thereon layer disposed thereon, include for example, implantable medical devices that are used for systemic treatment, as well as those that are used for the localized treatment of any tissue or organ. Non-limiting examples are tumors, organs including the heart, coronary and peripheral vascular system (referred to overall as “the vasculature”), the urogenital system, including kidneys, bladder, urethra, ureters, prostate, uterus and ovaries, eyes, ears, spine, nervous system, lungs, trachea, esophagus, intestines, stomach, brain, liver and pancreas, skeletal muscle, smooth muscle, breast, dermal tissue, cartilage, tooth and bone. As used herein, “treatment” refers to the reduction or elimination of symptoms associated with a disease or condition, or the substantial or complete elimination of a disease or condition. Subjects are vertebrate subjects, more typically mammalian subjects, including human subjects, pets, and livestock.

In some instances, the implantable medical device is a stent, wherein the surface are luminal, abluminal, or combination of those surfaces, and a layer of endosome-disrupting agent conjugated to at least one therapeutic agent is disposed on at least a portion of the substrate surface. Typically, the stent is an intravascular stent comprising an open lattice sidewall structure and designed for permanent implantation into a blood vessel of a patient. Examples include, an expandable stent, such as a self-expandable stent or balloon-expandable stent, having a tubular metal body having open ends and a sidewall structure having openings therein and a layer of surface-binding cell adhesion polypeptides disposed on at least a portion of the surface of the sidewall structure.

Substrate materials for the medical devices of the implantable medical devices disclosed herein can be selected from a range of biostable materials and biodisintegrable materials (i.e., materials that, upon placement in the body, are dissolved, degraded, resorbed, and/or otherwise removed from the placement site), including (a) organic materials (i.e., materials containing organic species, typically 50 wt % or more, for example, from 50 wt % to 75 wt % to 90 wt % to 95 wt % to 97.5 wt % to 99 wt % or more) such as polymeric materials (i.e., materials containing polymers, typically 50 wt % or more polymers, for example, from 50 wt % to 75 wt % to 90 wt % to 95 wt % to 97.5 wt % to 99 wt % or more) and biologics, (b) inorganic materials (i.e., materials containing inorganic species, typically 50 wt % or more, for example, from 50 wt % to 75 wt % to 90 wt % to 95 wt % to 97.5 wt % to 99 wt % or more), such as metallic materials (i.e., materials containing metals, typically 50 wt % or more, for example, from 50 wt % to 75 wt % to 90 wt % to 95 wt % to 97.5 wt % to 99 wt % or more) and non-metallic inorganic materials (i.e., materials containing non-metallic inorganic materials, typically 50 wt % or more, for example, from 50 wt % to 75 wt % to 90 wt % to 95 wt % to 97.5 wt % to 99 wt % or more) (e.g., including carbon, semiconductors, glasses and ceramics, which may contain various metal- and non-metal-oxides, various metal- and non-metal-nitrides, various metal- and non-metal-carbides, various metal- and non-metal-borides, various metal- and non-metal-phosphates, and various metal- and non-metal-sulfides, among others), and (c) hybrid materials (e.g., hybrid organic/inorganic materials, for instance, polymer/metallic-inorganic hybrids and polymer/non-metallic-inorganic hybrids).

Specific examples of inorganic non-metallic materials may be selected, for example, from materials containing one or more of the following: metal oxide ceramics, including aluminum oxides and transition metal oxides (e.g., oxides of titanium, zirconium, hafnium, tantalum, molybdenum, tungsten, rhenium, iron, niobium, and iridium); silicon; silicon-based ceramics, such as those containing silicon nitrides, silicon carbides and silicon oxides (sometimes referred to as glass ceramics); calcium phosphate ceramics (e.g., hydroxyapatite); carbon; and carbon-based, ceramic-like materials such as carbon nitrides.

Specific examples of metallic materials may be selected, for example, from metals such as gold, iron, niobium, platinum, palladium, iridium, osmium, rhodium, titanium, tantalum, tungsten, ruthenium, and magnesium, among others, and metal alloys such as those comprising iron and chromium (e.g., stainless steels, including platinum-enriched radio-opaque stainless steel), niobium alloys, titanium alloys, alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N), alloys comprising cobalt, chromium, tungsten and nickel (e.g., L605), alloys comprising nickel and chromium (e.g., inconel alloys), and biodisintegrable alloys including alloys of magnesium and/or iron (and their alloys with combinations of Ce, Ca, Zn, Zr and Li), among others.

Specific examples of organic materials include polymers (biostable or biodisintegrable) and other high molecular weight organic materials, and may be selected, for example, from suitable materials containing one or more of the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides, polyamidimides, polyesterimides, and polyetherimides; polysulfone polymers and copolymers including polyarylsulfones and polyethersulfones; polyamide polymers and copolymers including nylon 6,6, nylon 12, polyether-block co-polyamide polymers (e.g., Pebax® resins), polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinylacetate copolymers (EVA), polyvinylidene chlorides, polyvinyl ethers such as polyvinyl methyl ethers, vinyl aromatic polymers and copolymers such as polystyrenes, styrene-maleic anhydride copolymers, vinyl aromatic-hydrocarbon copolymers including styrene-butadiene copolymers, styrene-ethylene-butylene copolymers (e.g., a polystyrene-polyethylene/butylene-polystyrene (SEBS) copolymer, available as Kraton® G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene block copolymers such as SIBS), polyvinyl ketones, polyvinylcarbazoles, and polyvinyl esters such as polyvinyl acetates; polybenzimidazoles; ionomers; polyalkyl oxide polymers and copolymers including polyethylene oxides (PEO); polyesters including polyethylene terephthalates, polybutylene terephthalates and aliphatic polyesters such as polymers and copolymers of lactide (which includes lactic acid as well as d-,l- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of polylactic acid and polycaprolactone is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; polyurethanes; p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, and glycosaminoglycans such as hyaluronic acid; as well as blends and further copolymers of the above.

Examples of bifurcated stents and systems for delivery into vasculature include, but are not limited to, those shown and described in U.S. patent application Ser. No. 10/375,689, filed Feb. 27, 2003 and U.S. patent application Ser. No. 10/657,472, filed Sep. 8, 2003, both of which are entitled Rotating Balloon Expandable Sheath Bifurcation Delivery; U.S. patent application Ser. No. 10/747,546, filed Dec. 29, 2003 and entitled Rotating Balloon Expandable Sheath Bifurcation Delivery System; and U.S. patent application Ser. No. 10/757,646, filed Jan. 13, 2004 and entitled Bifurcated Stent Delivery System, the entire content of each being incorporated herein by reference. It should also be further noted that while stent 100 may be a standard “single vessel” stent such as is described above, or stent 100 may also be a bifurcated stent having a trunk or stem portion, with one or more leg portions and/or branch openings adjacent thereto. Such bifurcated stents and stent assemblies are well known in the art.

Use

Generally, an implantable medical device having endosome-disrupting agent conjugated to at least one therapeutic agent disposed on at least a portion of one substrate surface, may be configured to deliver one or more therapeutic agents to a delivery site, such as within the vessel or one or more areas adjacent thereto.

Application Methods

Typically, an endosome-disrupting agent conjugated to at least one therapeutic agent is disposed on at least a portion of an implantable medical device in an agent eluting coating or polymeric layer. The agent eluting coating will typically comprise, for example, from 1 wt % or less to 2 wt % to 5 wt % to 10 wt % to 25 wt % to 50 wt % or more of an endosome-disrupting agent conjugated to at least one therapeutic agent or of a mixture endosome-disrupting agents conjugated to therapeutic agents within the layer.

The agent eluting coating will also typically comprise, for example, from 50 wt % or less to 75 wt % to 90 wt % to 95 wt % to 97.5 wt % to 99 wt % or more of a single polymer or a mixture polymers within the layer. Polymers may biodegradable or biostable and may be selected, for example, from those described above for use in substrates, among others.

The thickness of the agent eluting coating may vary widely, typically ranging from 10 nm to 25 nm to 50 nm to 100 nm to 250 nm to 500 nm to 1 μm to 2.5 μm to 5 μm to 10 μm to 20 μm or more in thickness.

The agent eluting coating may be disposed on substrates using any suitable method known in the art. For example, where the layer contains one or more polymers having thermoplastic characteristics, the layer may be formed, for instance, by (a) providing a melt that contains polymer(s), therapeutic agent(s), and any other optional species desired and (b) subsequently cooling the melt. As another example, a layer may be formed, for instance, by (a) providing a solution or dispersion that contains one or more solvent species, polymer(s), therapeutic agent(s), and any other optional species desired and (b) subsequently removing the solvent species. The melt, solution or dispersion may be disposed on at least a portion of a substrate surface, for example, by extrusion onto the substrate, by co-extrusion along with the substrate, by roll-coating the substrate, by application to the substrate using a suitable application device such as a brush, roller, stamp or ink jet printer, by dipping the substrate, spray coating the substrate using spray techniques such as ultrasonic spray coating and electrohydrodynamic coating, among other methods. In certain instances, another surface of the substrate is masked to prevent the therapeutic-agent-eluting polymeric layer from being applied thereon.

### Example

### Synthesis of Endosome Disrupting Agent-Drug Conjugates

Poly(L-glutamic acid) (PG) was conjucated to Paclitaxel (PTx) via ester bonding (preferred PTx OH-group at position 2′). The conjugation was mediated by DCC (Dicyclohexyl carbodiimide) and CDI (N,N′-Carbonyldiimidazole).

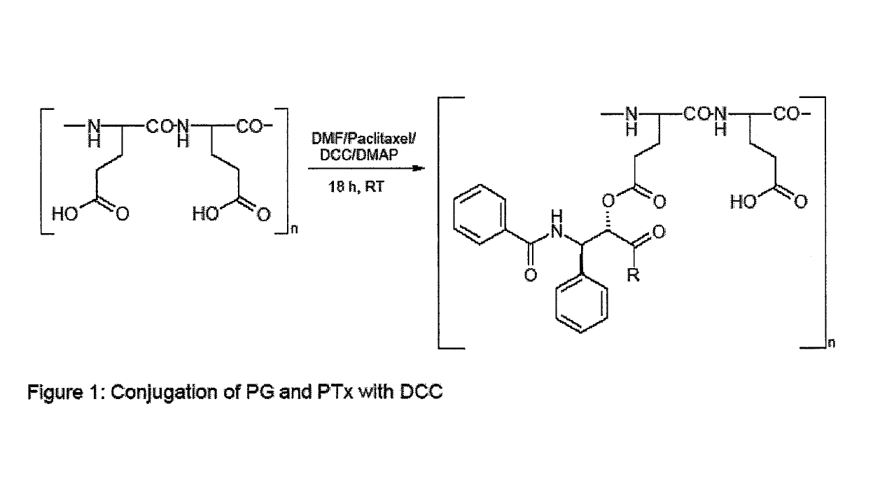

The reaction of DCC with the carboxylate group of the PG and the hydroxyl group of the PTx was carried out in N,N-dimethylformamide (DMF) to form an ester (published in U.S. Pat. No. 6,515,017). The reaction scheme is presented in FIG. 1. PG sodium salt (PG-Na) was obtained from Sigma Aldrich. We have used PG of a molecular weight (Mw) of 15.000-50.000 (P4761-1G) and of Mw of 50.000-100.000 (P4886-1G).

PG sodium salt (PG-Na) was first converted to PG in its proton form. The pH was adjusted to 2 using 0.2 M HCl and stirred over 2 h at room temperature (RT). The precipitate was collected and dialyzed over 3 days against water and finally lyophilized (3 days at 0.1 mbar at 30° C.). Then an amount of the dried PG (75 mg) was dissolved in appropriate amounts of DMF and added PTx (22 mg), DCC (15 mg) and dimethylaminopyridin (DMAP, 1-10 mg). The reaction at room temperature was allowed to proceed for 18 hours.

Thin layer chromatography was used to investigate the success of the conjugation. A small amount of the reaction mixture was diluted 1:10 with the solvent mixture (CHCl3/MeOH=10:1) to reduce side effects of DMF. A diluted solution of PTx in Methanol (RF-0.6) was used as a standard. To stop the reaction, the reaction mixture was poured into CH₂Cl₂ or CHCl₃ stabilized with amylen. The precipitate was washed with CHCl₃ and dried under vacuum. The sodium salt of the conjugate was obtained by dissolving the product in DMF and by dilution with a water-phase, for instance 0.5 M NaHCO3, 50 mM acetate buffer solution pH 5.6. The aqueous PG-PTx solution was dialyzed against distilled water (Mw cut off 3.5 kDa or 12 kDa) to remove low molecular weight contaminants. Finally we filtrated (0.8 μm), lyophilized the dialysate and obtained a white powder.

TABLE 1
Overview of reaction batches.

| | | | coupling | |
|---|---|---|---|---|
| | Polymer | Paclitaxel | reagent | Molar ratio |
| No | [mg; kDa] | [mg] | [mg] | PTx:Reagent:Polymer |
| Ca01 | N-acetyl-PG | 20 | DCC, 15 | 1:3.1:25 |
| | (75, 50-100) | | | |
| Ca02 | N-acetyl-PG | 7 | DCC, S | |
| | (25, 50-100) | | | |
| Ca03 | PG | 22 | DCC, 15 | 1:2.8:23 |
| | (75, 50-100) | | | (diverse purification |
| Ca04 | PG | 22 | DCC, 15 | steps) |
| | (75, 50-100) | | | |
| Ca05 | PG | 22 | DCC, 15 | |
| | (75, 50-100) | | | |
| B1 | PG | 40 | DCC, 39 | 1:4:10 |
| | (60, 15-50) | | | |
| B2 | PG | 40 | DCC, 39 | 1:4:10 |
| | (60, 50-100) | | | |
| B3 | N-acetyl-PG | 40 | DCC, 39 | 1:4:10 |
| | (60, 50-100) | | | |
| B5 | PG | 40 | DCC, 39 | 1:4:10 |
| | (60, 15-50) | | | |
| B6 | PG | 22 | DCC, 15 | 1:3.8:23 |
| | (75, 15-50) | | | |
| B23-27 | PG | 22 | DCC, 15 | 1:2.8:23 |
| | (75, 50-100) | | | |
| B29-33 | PG | 22 | DCC, 15 | 1:2.8:23 |
| | (75, 15-50) | | | |
| B34 | PG | 330 | DCC, 225 | 1:2.8:5 |
| | (250, 50-100) | | | |
| B35 | PG | 69 | DCC, 50 | 1:3:21, 6 |
| | (225, 50-100) | | | |
| B36 | PG | 22 | DCC, 15 | 1:2.8:23 |
| | (75 50-100) | | | |
| B37 | PG | 22 | DCC, 21, | 1:4:23 |
| | (75, 50-100) | | 3 | |
| B38 | PG | 22 | DCC, 30 | 1:5.6:23 |
| | (75, 50-100) | | | |
| B39 | PG | 22 | DCC, 15 | 1:2.8:23 |
| | (75, 15-50) | | | |
| B40 | PG | 22 | DCC, 21, | 1:4:23 |
| | (75, 15-50) | | 3 | |
| B41 | PG | 22 | DCC, 30 | 1:5.6:23 |
| | (75, 15-50) | | | |

All publications and patent applications in this specification are indicative of the level of ordinary skill in the art to which this invention pertains and are incorporated herein by reference in their entireties.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention without following the example embodiments and applications illustrated and described herein, and without departing from the true spirit and scope of the present invention, which is set forth in the following claims.

1-6. (canceled)

7. A medical device comprising:
a substrate that is expandable from a compressed state to an expanded state, and
a coating on said substrate wherein a drug agent and endosome-disrupting agent are incorporated into said coating.

8. The medical device of claim 7, wherein the medical device is a balloon.

9. The balloon of claim 8, wherein the drug agent is an antiproliferative drug.

10. The balloon of claim 8, wherein the drug agent is a restenosis-inhibiting agent.

11. The balloon of claim 8, wherein the drug agent is an immunosuppressant.

12. The balloon of claim 8, wherein the drug agent is rapamycin.

13. The balloon of claim 8, wherein the drug agent is everolimus.

14. The balloon of claim 8, wherein the endosome disrupting agent is a GALA peptide.

15. The balloon of claim 14, wherein the GALA peptide comprises at least 10 amino acids.

16. The balloon of claim 15, wherein the GALA peptide comprises 20 to 100 amino acids.

17. The balloon of claim 16, wherein the GALA peptide comprises the polypeptide WEAALAEALAEALAEHLAEALAEALEALAA (SEQ ID NO: 1).

18. The balloon of claim 8, wherein the endosome disrupting agent is a KALA peptide.

19. The balloon of claim 18, wherein the KALA peptide comprises the polypeptide Trp-Glu-Ala-Lys-Leu-Ala-Lys-Ala-Leu-Ala-Lys-Ala-Leu-Ala-Lys-His-Leu-Ala-Lys-Ala-Leu-Ala-Lys-Ala-Leu-Ala-Lys-Ala-Leu-Lys-Ala-Cys-Glu-Ala (SEQ ID NO:2).


SEQUENCE LISTING (2 sequences)

SEQ ID NO: 1 - protein, 30 aa, UNKNOWN
  Synthetic peptide
WEAALAEALAEALAEHLAEALAEALEALAA

SEQ ID NO: 2 - protein, 34 aa, UNKNOWN
  Synthetic peptide
WEAKLAKALAKALAKHLAKALAKALAKALKACEA